(19) United States
(12) Patent Application Publication
(10) Pub. No.: US 2005/0234473 A1
(43) Pub. Date: Oct. 20, 2005

(54) Phacoemulsification probe with tip shield

(76) Inventor: JAIME ZACHARIAS, SANTIAGO (CL)

Correspondence Address: JAIME ZACHARIAS, AV. LUIS PASTEUR 5917 - VITACURA, SANTIAGO, 6670775, CL

(21) Appl. No.: 10/907,588

(22) Filed: Apr. 7, 2005

Related U.S. Application Data
(60) Provisional application No. 60/521,375, filed on Apr. 14, 2004.
(60) Provisional application No. 60/521,853, filed on Jul. 11, 2004.

(51) Int. Cl. A61F 9/00

(52) U.S. Cl. 606/107

(57) ABSTRACT

A surgical probe with a tip shield (10) for lens removal by vibratory surgical instruments. The probe with tip shield (10) including a tip cap (60) disposed as a membrane covering the distal end (16) surface of a vibratory probe (52) to act to protect the lens capsule during operation. Direct contact between the metallic vibratory probe (52) and the lens capsule (68) is avoided by the interposed tip cap (60) even when the capsule (68) is aspirated by the central aspiration opening (64). Vibratory energy applied to probe tip (50) is transmitted to the lens tissue across the walls of tip cap (60) to provide protection of the lens capsule (68) while effectively fragmenting and liquefying the tissue by emulsification.

### BACKGROUND: CROSS REFERENCE TO RELATED APPLICATIONS

This application claims the benefit of Provisional Patent Application Ser. 60/521,375 filed Apr. 14, 2004 titled “Safe sleeve for surgical instrument” and of Provisional Patent Application Ser. 60/521,853 filed Nov. 07, 2004 titled “Phacoemulsification probe with tip shield for surgical instrument”.

### BACKGROUND: FIELD OF THE INVENTION

The invention generally pertains to phacoemulsification instruments that are used to remove the lens of the eye, and more particularly, to an oscillatory probe incorporating a resilient tip shield, the probe attached to the forefront of a vibratory surgical instrument.

### BACKGROUND: DISCUSSION OF PRIOR ART

A common problem in the state of the art removal of the lens tissue during cataract surgery is the potential for lens capsule rupture by direct contact by the bare metallic tip of the phacoemulsifier probe with the lens capsule.

Accidental rupture of the lens capsule is an untoward surgical event associated with several intra-operative and post-operative complications.

Up to date, it has been accepted that providing a phacoemulsifier probe with a tip having sharp cutting edges favors the efficiency of the phacoemulsification process, particularly when operating the hard cataractous lenses.

Unfortunately, bare metal probes frequently rupture the lens capsule by direct contact because of the action of the sharp cutting edges or by the presence of tiny burrs and spurs.

Refractive lens exchange procedures have become increasingly popular to adjust the optics of the eye by replacement of the natural lens with a suitable intraocular lens.

Accidental capsule rupture in the refractive lens exchange population is highly undesirable because it adds the problem of preventing the implantation of the programmed intraocular lens.

Aimed to provide a vibratory probe that is less aggressive to the lens capsule, the published United States Patent Application #20040267211 filed Mar. 23, 2004 titled “Phacoemulsification needle” presented by T. Akahoshi proposes a probe with a bulb shaped metallic tip.

The fundament for this application is the fact that in cases where no cataract is present, the lens of the eye is softer, allowing an expedite removal with a blunt metal probe without cutting edges.

However, it is the experience of this author, who has performed thousands of cataract procedures, that any metallic vibratory probe will develop undetectable burrs and spurs as a consequence of normal use in a surgical environment.

For example, burrs and spurs will develop in the vibratory probe tip surface during operation by contact with any other hard structure such as the auxiliary instruments jointly used in the lens removing process.

For this reason the blunt nature of the cited reference for a metallic probe is not reliable as it will not prevent capsule rupture because burrs or spurs that can contact the lens capsule can develop on the tip surface during normal use producing a rupture.

### OBJECTS AND ADVANTAGES

We have found no prior art where the vibratory activity from a phacoemulsifier probe is deployed to the lens tissue across a capsule safe shield attached to the probe tip to protect the lens capsule from accidental rupture caused by direct contact with the probe metal while effectively removing the lens tissue.

It is a known fact for cataract surgeons that covering the metallic surface surrounding the aspiration port of a passive irrigation-aspiration cannula with an elastomer membrane makes accidental capsule rupture highly unlikely even when aspirating the lens capsule using high vacuum.

Phaco-refractive procedures aimed to correct a refractive defect by exchanging the lens of the eye can be successfully performed using the phacoemulsifier probe with incorporated elastomer tip shield of the present invention.

Furthermore, using this system, when the lens capsule is aspirated by the aspirating opening at the distal end of the phacoemulsification probe it will preserve its integrity.

Therefore, a primary object of the present invention is to provide a capsule safe vibratory probe by combination of the metal probe tip with an elastomer shield disposed to cover the metal surface of the probe in a way that the metal of the probe tip is isolated from the lens capsule during operation.

Another object of the present invention is to provide a surgical probe with tip shield that allows expedite and safe aspiration of the residual lens cortical material, reducing the need to use a separate instrument such as an irrigation-aspiration cannula.

These and other objects and advantages of the present invention will become apparent from the subsequent detailed description of the preferred embodiment and the appended claims taken in conjunction with the accompanying drawings.

### DESCRIPTION OF DRAWINGS

FIG. 1 represents prior art with a cut-away diagram of a human eye with a conventional probe penetrating an incision through the cornea of an eye maintaining an exposed metallic tip of the phacoemulsifier probe.

FIG. 2 represents prior art showing a longitudinal hemi-sectional view of the tip of a phacoemulsification probe with sharp cutting edges.

FIG. 3 is a cut-away diagram of a human eye with a probe incorporating the current invention. This embodiment provides a tip shield as an extension from an irrigation sleeve. The tip shield transmits the vibratory energy for emulsifying the lens and removes the liquefied particles through its hollow interior while protecting the lens capsule by impeding direct contact with bare metal.

FIG. 4 is an isometric view of a typical embodiment where the tip shield is incorporated into an infusion sleeve that is placed over the metal probe.

FIG. 5 is an isometric view of a phacoemulsifier probe.

FIG. 6 is a detailed lateral isometric view of the distal end of a phacoemulsifier probe with blunt tip for use in combination with a tip shield.

FIG. 7 is a cross-sectional view taken along lines 2-2 of FIG. 4 showing the distal end portion of a probe tip with elastomer shield.

FIG. 8 is a cross-sectional view taken along lines 2-2 of FIG. 4 showing the distal end of an irrigating sleeve providing the tip shield for use in combination with a metal probe.

FIG. 9 is a cross-sectional view taken along lines 2-2 of FIG. 4 showing the probe and accompanying tip shield with ribs and extending from a covering sleeve.

FIG. 10A is a cross-sectional view taken along lines 2-2 of FIG. 4 showing the probe and accompanying tip shield extending from an irrigating sleeve the tip shield including an internally anchored axial tensing fiber extension.

FIG. 10B is a cross-sectional view taken along lines 2-2 of FIG. 4 showing the probe and accompanying tip shield extending from an irrigating sleeve the tip shield including an internal axial tensing extension with a non-occlusive dilatation acting as an internal anchor point.

FIG. 11A is a cross-sectional view taken along lines 2-2 of FIG. 4 showing the probe incorporating a tip shield attached with button-like extensions inserted through perforations in the metal probe wall.

FIG. 11B is a detailed isometric lateral view of the distal end of a blunt phacoemulsifier probe with accompanying tip shield attached with buttons placed in perforations in the probe wall.

FIG. 12A depicts a side view of the distal end of a phacoemulsifier probe with the accompanying tip shield fixated to the same probe structure by fusion or adhesion of the shield material through perforations in the probe wall.

FIG. 12B depicts a longitudinal cross sectional view of the phacoemulsifier probe of FIG. 12A with the accompanying tip shield fixated to the same probe by fusion of the external and bore portions of the probe shield through perforations in the probe wall.

FIG. 13A is a longitudinal hemi-sectional view of the tip shield portion of the present invention designed to be attached by elastic fitting into the flared shaped distal end of the probe portion of the present invention.

FIG. 13B depicts a side view of the probe tip shield of FIG. 13A installed on the flared phacoemulsifier probe.

FIG. 13C is a cross sectional view of the assembled distal end of the flared probe with attached tip shield of FIG. 13A in position for operation.

FIG. 14 is a cross sectional view of the phacoemulsifier probe with accompanying tip shield of the present invention illustrating the isolating action of the tip shield portion impeding direct contact between the metal of the probe portion and the lens capsule.

FIG. 15A is a partial cut away view of the probe with tip shield showing a mesh of thin fibers embedded in the elastomer in the zone that covers the tip area of the probe.

FIG. 15B depicts a front axial view of the tip shield showing the embedded mesh of fibers that cover the rim of the probe tip distal end.

### LIST OF REFERENCE NUMERALS

FIGURE NUMERALS: 10 probe with tip shield, 13 probe longitudinal bore, 14 probe body, 15 probe proximal end, 16 probe distal end, 17 probe engaging means, 18 threads, 19 sleeve, 20 hollow sleeve body, 22 sleeve proximal end, 24 sleeve distal end, 26 surgical instrument, 30 sleeve tubular shank, 32 sleeve truncated cone contour, 40 iris, 42 lens tissue, 44 cornea, 46 small incision, 48 sclera, 50 probe vibratory tip, 51 sharp probe edges, 52 phacoemulsifier probe, 60 tip shield, 61 annular zone, 62 tip shield annular stricture, 63 bore portion of shield, 64 central aspiration opening, 66 sleeve fluid ports, 67 thin fibers, 68 lens capsule, 69 aspirated lens capsule, 70 grooves, 72 internal metal surface, 74 tip rim surface, 76 external metal surface, 80 stiffening ribs, 82 internal tensing fiber, 83 non-occlusive stopper, 84 internal openings, 86 perforations, 87 aspirate conducts, 88 buttons, 89 fused shield walls, 90 flared tip, 92 elastic stricture.

### SUMMARY

An improved capsule safe vibratory probe for removing the lens of the eye with attached shield disposed to cover the probe tip surface in a way to prevent direct contact between the metal of the probe tip and the lens capsule during a lens removing surgery while effectively transmitting vibratory energy to disrupt the tissue composing the lens of the eye.

### DESCRIPTION OF INVENTION

The invention is a phacoemulsifier probe including a tip shield, the probe placed on the forefront of a phacoemulsification instrument. The instrument consists of a hand piece containing a piezoelectric, magnetic or magnetostrictive mechanism which, using electric pulses, activates in a vibratory fashion the hollow probe with attached tip shield of the present invention.

When the probe tip contacts an eye lens, vibration emulsifies the surrounding lens tissue and the displaced tissue particles are drawn under negative pressure into the hollow probe. Simultaneously, a saline solution may be delivered between a sleeve and the outer wall of the probe down to irrigation ports for cooling and irrigation purposes.

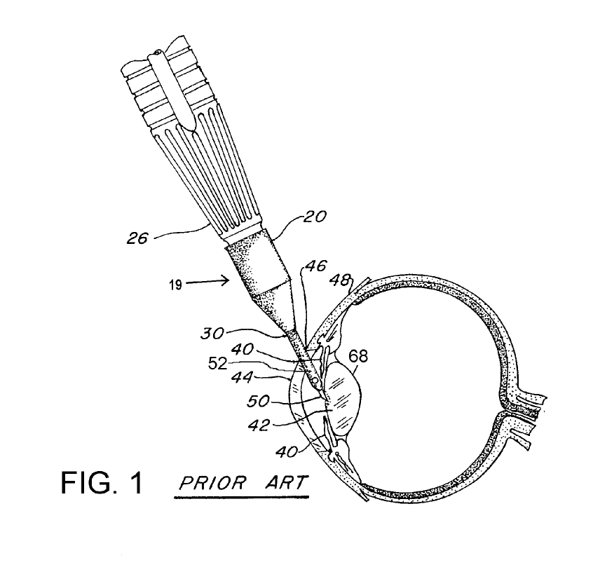

FIG. 1. illustrates a surgical instrument 26 and a prior art probe 10 penetrating the cornea 44 through a surgical incision 46 with the probe 52 of the instrument 26 engaging the ocular lens 42 with the exposed bare metal of the sharp probe tip 50 potentially touching, aspirating and rupturing the lens capsule 68. Iris 40 is usually pharmacologically dilated. An irrigating sleeve body 19 having a body 20, a proximal end 22 and sleeve tubular shank 30 conduct cooling and irrigation fluid into the eye during operation.

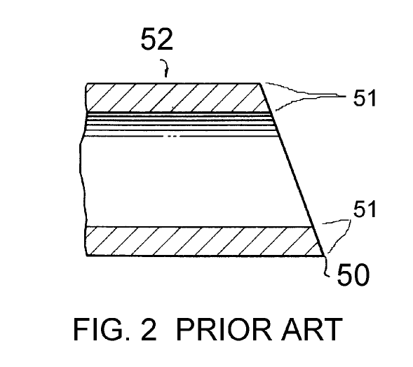

FIG. 2 is a prior art probe tip in longitudinal cross section with detailed view of the sharp edges 51 of the vibratory tip 50 of a typical phacoemulsifier probe 52 susceptible to cause rupture of the lens capsule 68 by direct contact or aspiration.

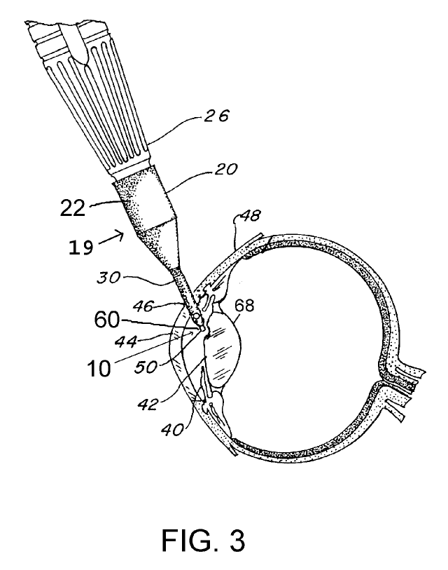

In FIG. 3 the shielded probe 10 of the present invention has attached sleeve 19 that provides a tip shield 60 and is inserted into a small surgical incision 46. In this configuration the vibratory tip 50 can only contact tissues 42, 40, 68 across the walls of the elastomer tip shield 60. The surgical sleeve 19 may deliver irrigating fluid for cooling purposes and for replacing intraocular fluid through ports 66.

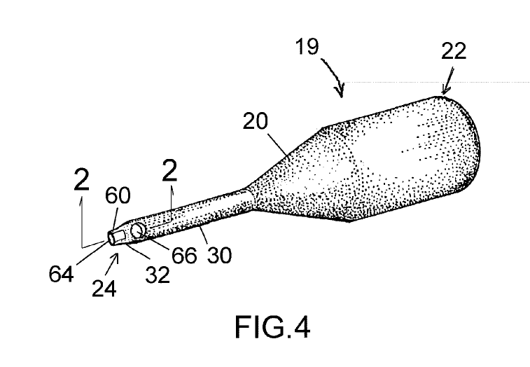

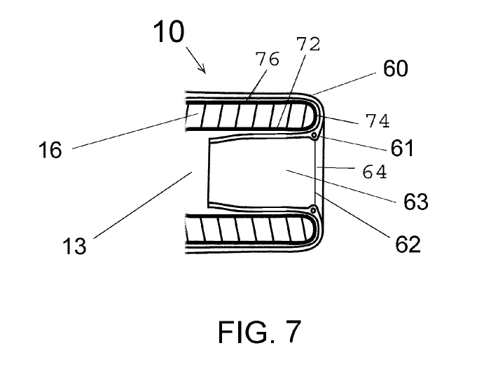

A preferred embodiment of the vibratory probe 52 with tip shield 10 is shown in FIG. 4 to 8 where the tip shield 10 is provided by an extension of a sleeve 19 including hollow sleeve body 20 with proximal end 22 and distal end 24.

As detailed in FIG. 4 the proximal end of sleeve body 20 has a configuration that is adaptable to be removably attached to the forefront of a surgical instrument 26 for phacoemulsification and aspiration of a cataract lens of a human eye. A plurality of internal diametric ridges, threads or the like may be formed within the proximal end 22 for interfacing with, and gripping onto, the surgical instrument 26. The distal end 24 of the body 20 is formed into hollow tubular shank 30 with a truncated cone contour 32 on its extremity.

The sleeve 19 is preferably constructed of a material selected from a group consisting of polimethylsiloxane, natural or synthetic latex, hydroxymethylmethacrylate, polymide, polymethylmethacrylate, polyethylene, polyester, polystyrene, polypropylene, polytetrafluorethylene, polyurethane, ethylene-vinyl-acetate.

The tip shield portion 10 is preferably constructed of an elastomer material selected from a group consisting of polimethylsiloxane, natural or synthetic latex and other resilient elastomer compounds.

Further the elastomeric material should be pliable, resilient and non-absorbent. As shown in FIGS. 4 through 10, in this embodiment tip shield 60 cooperatively combines with probe 52 tip. Tip shield 60 is integrally formed with shank 30 and truncated cone 32 and extends axially forward, bends around tip 50 rim and reflects inward and backward in a way to continuously cover the metallic surface of probe distal end 24 in an amount to prevent contact between the lens capsule an the bare metal of probe 52. As shown in FIG. 4 a probe tip aspiration opening 64 is maintained with a diameter preferably ranging between 0.1 and 1.5 mm.

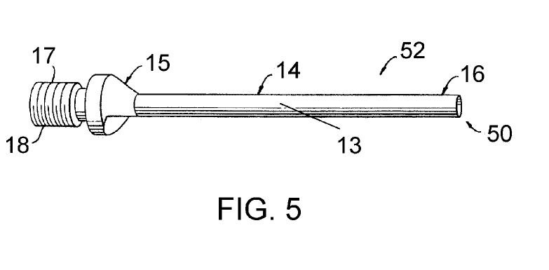

FIG. 5 illustrates the phacoemulsifier probe part 52 of the present invention generally comprising a body 14 and having a proximal end 15 and a distal end 16. Means 17, disposed at the proximal end 15 of the body 14, are provided for engaging in a conventional manner, a vibratory hand piece 26. For example, the proximal end 15 may include threads 18 for enabling screwing of the probe 52 onto a reciprocating portion of the hand piece 26. The probe 52 further comprises a tip 50, disposed at the distal end 16 of the body 14.

Also included are aspiration means, defined by a longitudinal bore 13 through said body portion 14 and tip portion 50 provided for enabling passage of dislodged tissue through the probe 52 such that tissue debris and fluids may be removed from the surgical site in a conventional manner, for example, by a remote source of suction.

The phacoemulsifier probe with tip shield 10 of the present invention is arranged with tip shield 60 covering the distal end 16 of phacoemulsifier probe 52. The rim of tip 50 of phacoemulsifier probe 52 is preferably blunt to avoid sharp angles, burrs or edges that can damage and perforate the surrounding thin walled tip shield 60.

A tip shield 60 of the present invention 10 installed in the emulsification probe 52 covers the vibratory tip 50 in a way that the vibratory energy is transmitted from probe 52 across the elastomer tip shield 60 to the lens material. This novel arrangement provides lens capsule protection and at the same time delivers adequate amounts of vibratory energy across the walls of tip shield 60 to effectively disrupt and aspirate the lens material 42 through probe central opening 64.

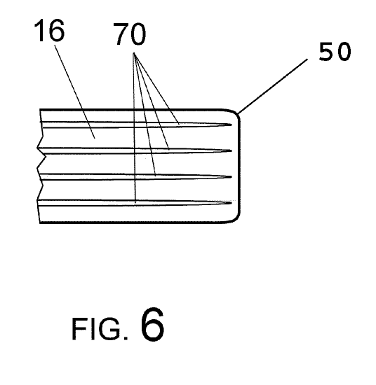

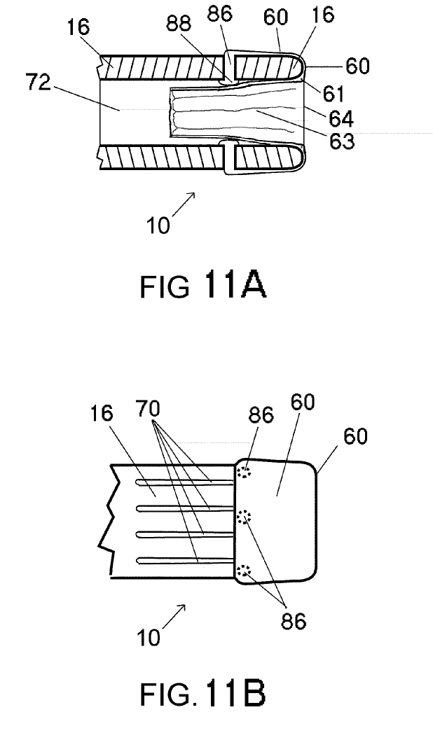

As shown in FIGS. 6 and 11B a plurality of preferably longitudinal grooves 70 can be incorporated over the external, rim and internal surface of the distal end 16 of probe 52 to allow the entrance and circulation of fluid between the contacting surfaces of metal probe distal end 16 and the overlying elastomer tip shield 60 surface to reduce heat buildup by friction during vibratory activity.

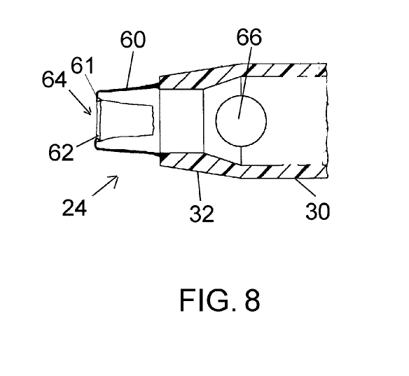

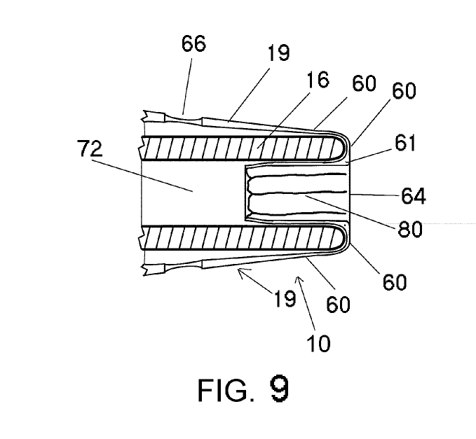

Best detailed in FIGS. 8 and 9 tip shield 60 can include a distal end annular stricture 62 to avoid slippage of tip shield 60 in proximal direction over the external walls of probe 52 exposing the bare metal to the lens capsule when tension is applied. This distal end annular stricture 62 can incorporate a less elastic annular zone 61 that can be formed by a thickening of the same elastomer material or by the inclusion of a different less distensible material.

A differential polymerization technique can be used during tip shield 60 manufacture to selectively produce a less distensible portion at the stricture 62 level. Tip shield 60 covers an external surface 76 and a tip rim surface 74 of probe distal end 16.

Preferably an internal portion 63 of tip shield 60 extends proximally covering the internal metal surface 72 of distal end 16 of probe 52 in an axial length typically between 0.1 and 5 mm, to prevent direct contact between lens capsule 68 and internal metal surface 72 of probe tip 50.

Tip shield 60 covers probe 52 tip 50 metal surfaces while preserving central aspiration opening 64 to allow proper aspiration of fluid and lens debris.

FIG. 9 illustrates an embodiment of a probe with tip shield 10 where tip shield 60 is provided by an irrigating sleeve 19. The shield disposed as a membrane continuously covers the external, rim and internal surface of probe 50 tip 52 distal end 16. The internal surface covering portion 63 of tip shield 60 can include a plurality of stiffening axial ribs 80 to avoid inversion of the shield membrane during reflux operation through the phacoemulsifier probe longitudinal bore 13. Stiffening ribs 80 are typically conformed by spaced thickenings of tip shield 60 composing elastomer. Stiffening ribs 80 can also be conformed by embedded materials of different rigidity.

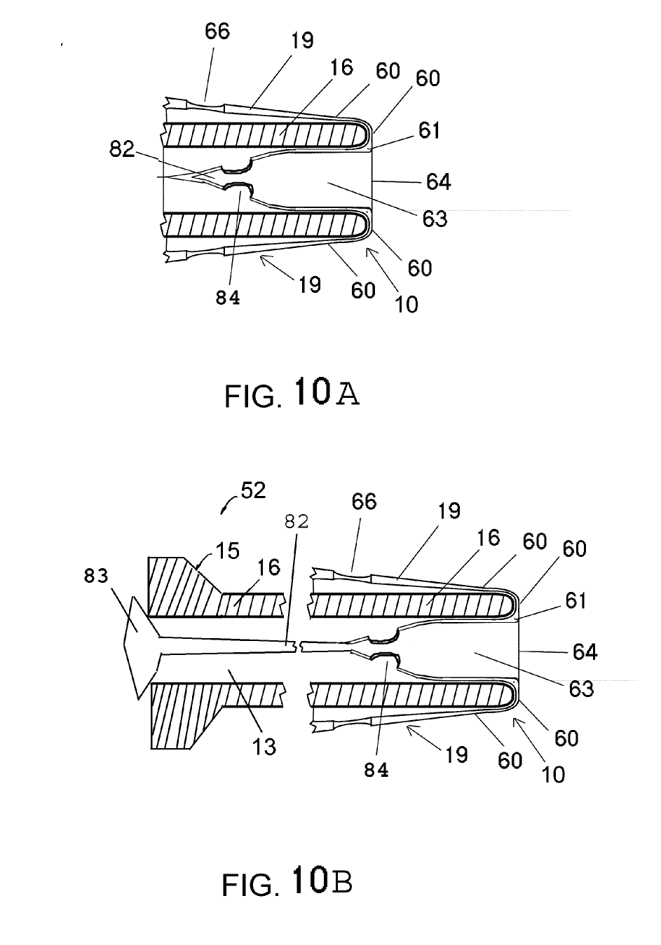

FIGS. 10A and 10B illustrate one embodiment where an internal tensing member 82 is attached to the internal aspect of tip shield 60 to operate with tension applied in a way to reduce tip shield 60 thickness and better adjust shield 60 membrane to the underlying probe tip 50 rim. A proximal stopper 83 can anchor internal tensing fiber 82 to the proximal end 15 of probe 52, preferably near a node of the vibratory activity where oscillations have reduced amplitude. This embodiment considers a proper path for the aspirated fluid and debris through internal openings 84 and aspirate passages 87.

FIGS. 11A and 11B illustrate other embodiment of the probe with tip shield 10 where tip shield 60 is directly anchored to the underlying probe 52 distal end 16 using perforations 86 and buttons 88 traversing the probe walls.

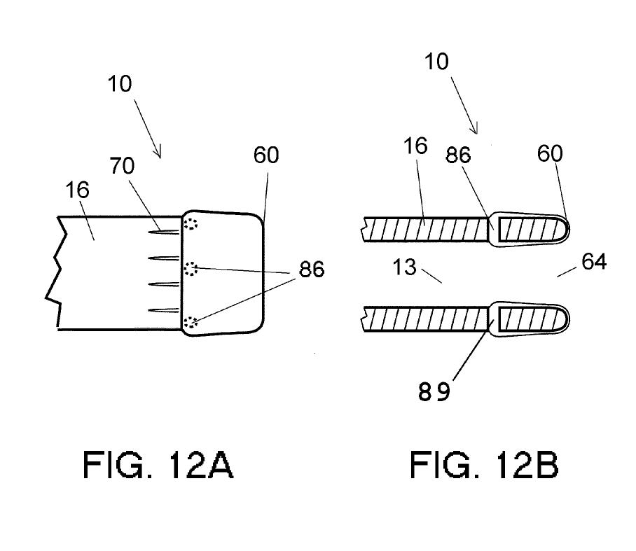

FIGS. 12A and 12B illustrate still another embodiment of the probe with tip shield 10 where tip shield 60 is directly anchored to the underlying probe 52 distal end 16 using perforations 86 that allow adhesion or fusion of the external and internal aspects of the tip shield 60 for fixation purposes.

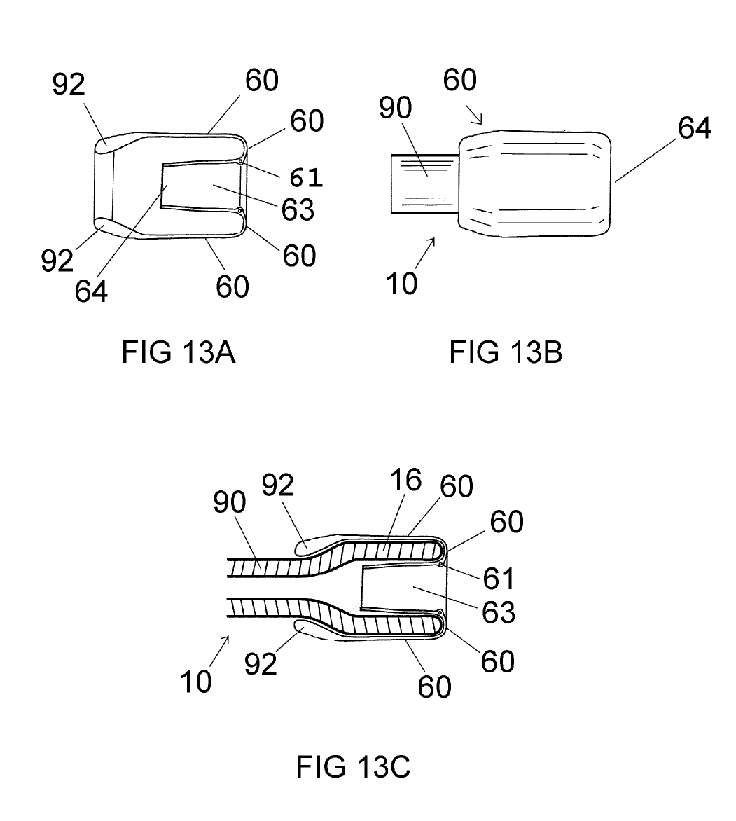

FIGS. 13A, 13B and 13C illustrate still another embodiment of the probe with tip shield 10 where tip shield 60 is directly anchored to an underlying flared tip probe 90 taking advantage of the proximal narrowing of the wide probe distal end 16. A proximal elastic stricture 92 keeps tip shield 60 properly attached to probe 52 during operation.

FIGS. 11 though 13 depict embodiments of the probe with tip shield 10 where tip shield 60 can be directly attached to probe 14 unrelated to an irrigating sleeve. This alternative embodiment has been purposely designed to allow the incorporation of the benefits of the current invention into the recently introduced bimanual phacoemulsification technique, where a second instrument carries the irrigation fluid through a secondary incision.

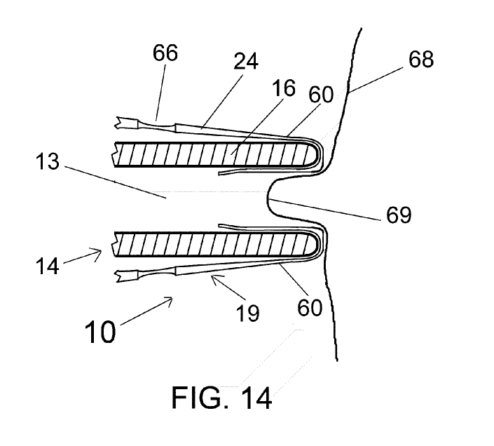

FIG. 14 shows a probe 52 with the tip shield 60 properly disposed to isolate the external metal surface 76, tip rim surface 74 and the internal metal surface 72 of vibratory probe tip 50 from direct contact with lens capsule 68.

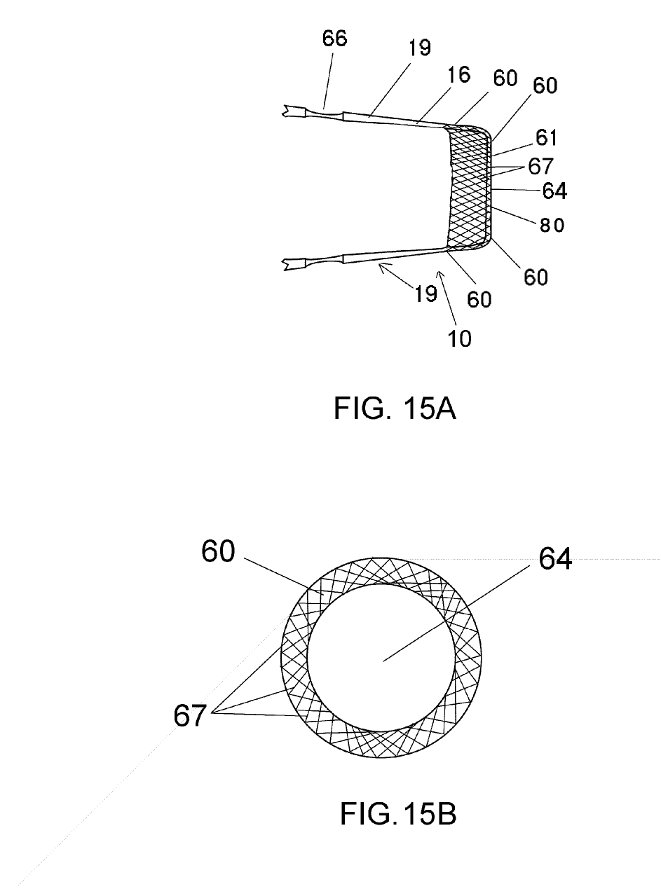

FIGS. 15A and 15B illustrate the incorporation of a mesh of fibers 67 into the tip shield 60 portion covering the tip of probe 52. This mesh of fibers 67 protects the tip shield 60 membrane from extended rupture that can expose the bare metal to the lens tissues. Perforation of tip shield 60 can occur when vibratory activity is applied through probe 52 and another instrument inside the eye is touched across shield 50 membrane.

The mesh of embedded fibers 67 prevents that a small puncture or tear of the shield membrane may expand and expose the metallic surface of probe tip 50. Embedded fibers 67 can be composed of strong lightweight materials such as kevlar, prolene, polypropilene, perlon and others.

In this way the probe with tip shield 10 of the present invention significantly reduces the risk of lens capsule rupture by separating the metal probe distal end 16 from the capsule 68 by elastomer membrane 60.

### OPERATION OF INVENTION

A lens extraction surgical procedure involves introduction into the eye of the probe distal end 16 putting the probe tip with shield 10 in direct contact with the lens tissue. The operator activates irrigation, aspiration and probe vibratory activity typically using a foot-pedal.

During operation, surgical instrument 26 transmits vibratory activity to the attached probe with tip shield 10. Vibratory tip 50 oscillates typically in a range of 1 to 200 microns. Vibratory tip 50 transmits the vibratory activity to the overlying tip shield 60, which is in direct contact with the lens tissue.

The transmitted vibratory activity is applied to the lens tissue producing fragmentation and liquefaction allowing aspiration through central aspiration opening 64. At the same time, tip shield 60 is arranged over vibratory tip 50 in a way to isolate the metal parts of probe distal end 16. In this way any sharp edges, burrs and spurs on the surface of vibratory tip 50 cannot contact and rupture the lens capsule 68.

Grooves 70 allow fluid penetration in the virtual space created by the probe tip 50 and the overlying elastomer shield 60. This fluid reduces friction between sliding parts preventing dangerous heat buildup. Tip shield annular stricture 62 and inelastic ring 61 operate to prevent back sliding of tip shield 60 and metal exposure to the lens capsule 68. Stiffening ribs 80 operate to avoid inversion and exteriorization of the inner aspect of tip shield 60 during reflow operations of the aspiration source that drives vacuum in aspiration opening 64.

During operation, optional embedded fibers 67 increase resistance of tip shield 60 to rupture and expose metal to the lens capsule as a consequence of eventual perforations.

### CONCLUSIONS, RAMIFICATIONS AND SCOPE OF INVENTION

The present invention has been used with advantage in eye surgery observing that the lens capsule 68 has resisted direct contact with the shielded probe 10 without rupture using high vacuum levels and vibratory activity.

Another advantage is that probe tip 50 with tip shield 60 of the present invention 10 can be used to aspirate the residual lens cortical material touching lens capsule 68 for cleaning purpose without rupture of the lens capsule 68 during these maneuvers as it would occur when the bare metal tip 50 is used.

While the present invention has been described in complete detail and pictorially shown in the accompanying drawings, it is not to be limited to such details, since many changes and modifications may be made to the invention without departing from the spirit and scope thereof.

For example, different modalities of vibratory activity can be applied. Ultrasonic, sonic, axial and rotational oscillatory modalities can all be used alone or in combination to drive the probe with shield 10 of the present invention.

Orientation of the central aspiration port 64 can be disposed at different angles relative to the probe main axis.

Location of the central aspiration port 64 can be vary to include the sidewalls of the probe distal end 16.

The number of aspiration ports can be expanded beyond one to include a plurality of aspiration ports 64.

Hence, it is described to cover any and all modifications and forms which may come within the language and scope of the appended claims.

1. A probe with attached tip shield for a phacoemulsification surgical instrument for removal of the lens of the eye with improved capsule safety properties comprising:
a. a hollow probe attached to the forefront of a phacoemulsification instrument,
b. a probe tip shield,
c. attaching means to hold said shield in contact with the tip of said vibratory probe, whereby said probe with attached tip shield is safe for the lens capsule while transmitting vibratory energy to effectively fragment and liquefy the lens tissue.

2. The probe with attached tip shield for a phacoemulsification surgical instrument as recited in claim 1 wherein said tip shield isolates the metal portions of said probe tip from the eye tissues.

3. The probe tip shield for a phacoemulsification surgical instrument as recited in claim 1 wherein said tip shield is supplied as an extension of an irrigating sleeve.

4. The probe tip shield for a phacoemulsification surgical instrument as recited in claim 1 wherein said tip shield uses attaching means disposed to attach said shield to the body of said phacoemulsifier probe.

5. The probe with tip shield for a phacoemulsification surgical instrument as recited in claim 1 wherein said tip shield is constructed using an elastomer material.

6. The sleeve with tip shield for a phacoemulsification surgical instrument as recited in claim 3 wherein said sleeve is constructed using at least one thermoplastic material selected from a group consisting of polimethylsiloxane, hydroxymethylmethacrylate, polymide, polymethylmethacrylate, polyethylene, polyester, polystyrene, polypropylene, polytetrafluorethylene, polyurethane, and ethylene-vinyl-acetate.

7. The probe tip shield for a phacoemulsification surgical instrument as recited in claim 1 wherein said shield is composed at least by one pliable elastomeric material selected from a group consisting of polimethylsiloxane, natural or synthetic latex and other resilient elastomer compounds.

8. The probe with tip shield for a phacoemulsification surgical instrument as recited in claim 1 wherein said probe tip shield incorporates materials of different elasticity.

9. The probe with tip shield for a phacoemulsification surgical instrument as recited in claim 1 wherein said tip shield has an annular stricture disposed at the probe tip having a diameter equal or smaller than that of the distal end of the vibratory probe acting as a stopper to avoid slippage of the tip shield over external walls of said probe.

10. The annular stricture as recited in claim 9 wherein said tip shield has an annular stricture that is located in front of the distal end of said phacoemulsification probe permitting an internal portion of said tip shield to continue covering the internal surface of the distal end of said probe.

11. The annular stricture as recited in claim 9 wherein said stricture incorporates a different material less distensible than the material composing the rest of the tip shield body.

12. The annular stricture as recited in claim 9 wherein said stricture is designed with secondary appendages to cover the internal surface of the distal end of the vibratory probe.

13. The annular stricture as recited in claim 9 wherein said annular stricture is designed to sustain axial tension applied to the tip shield distal end avoiding slippage and direct exposure of the metallic probe tip rim to the ocular tissues.

14. The probe with tip shield for a phacoemulsification surgical instrument as recited in claim 1 wherein said tip shield has a final operable thickness in the probe tip surface ranging between 0.001 mm and 0.500 mm.

15. The probe with tip shield as recited in claim 14 wherein the final operable thickness of said tip shield occurs in a natural state with no tension being applied.

16. The probe with tip shield as recited in claim 14 wherein the final operable thickness of said tip shield is obtained by elongation of the material by axial proximal tension of the proximal end of said tip shield towards the instrument hand-piece over the instrument vibratory probe distal end.

17. The probe with tip shield as recited in claim 14 wherein the final operable thickness of said tip shield is obtained by the distension of the composing material of said tip shield resulting from the introduction of a phacoemulsifier probe distal end of bigger diameter than that of said tip shield in natural resting conditions.

18. The probe with tip shield as recited in claim 14 wherein said tip shield membrane incorporates a mesh of strong fibers such as kevlar in the area corresponding to the tip rim of the underlying phacoemulsification probe.

19. A method for protecting the lens capsule during surgical removal of the lens of the eye using a vibratory probe comprising the steps of:
a. Providing a hollow vibratory probe having a tip,
b. Providing a shield disposed at the forefront of said vibratory probe in a way to isolate the tip surface of said probe that can otherwise contact the lens capsule,
c. Introducing said shield covered vibratory tip through a surgical incision into an eye,
d. Positioning said shield covered vibratory tip in close contact with the lens material,
e. Applying vibratory energy to said probe in a way that said vibratory energy is transmitted across said shield to effectively fragment and liquify the contacted lens material,
f. Applying vacuum to said probe central aspiration port to remove the liquefied lens material, whereby said shield covered vibratory probe is effective to reduce the chance of rupture of said lens capsule when contact between said lens capsule and said shield covered vibratory tip occurs.
